(19) United States
(12) Patent Application Publication
(10) Pub. No.: US 2017/0297984 A1
(43) Pub. Date: Oct. 19, 2017

(54) ETHYNYLATION OF POLYUNSATURATED ALDEHYDES AND KETONES

(71) Applicant: DSM IP ASSETS B.V., Heerlen (NL)

(72) Inventors: Werner BONRATH, Kaiseraugst (CH); Jan SCHUETZ, Kaiseraugst (CH); Thomas NETSCHER, Kaiseraugst (CH); Bettina WUESTENBERG, Kaiseraugst (CH)

(21) Appl. No.: 15/518,080

(22) PCT Filed: Oct. 15, 2015
(86) PCT No.: PCT/EP2015/073868

§ 371 Date: Apr. 10, 2017

(30) Foreign Application Priority Data
Oct. 16, 2014 (EP) 14189262.0

(51) Int. Cl. C07C 29/38; C07C 2601/16

(52) U.S. Cl. CPC C07C 29/38; C07C 2601/16

(57) ABSTRACT

The present invention relates to a catalytic process for the ethynylation of specific poly-unsaturated aldehydes and ketones.

The present invention relates to a catalytic process for the ethynylation of specific poly-unsaturated aldehydes and ketones.

Polyunsaturated aldehydes and ketones in the context of the present invention are aldehydes and ketones wherein the aldehydes and ketones comprise at least two C—C double bonds. In some embodiments of the present invention the C═C double bonds can also be conjugated.

Polyunsaturated aldehydes and ketones (with conjugated C═C double bonds), such as the α,β-unsaturated ones, are quite stable compounds.

The reaction products, which are the outcome of the process according to the present invention are useful intermediates in the production of important organic compounds (such as for example vitamin A or carotenoids).

Known ethynylations of specific polyunsaturated aldehydes and ketones are stoichiometric processes, such as for example the Grignard reaction. This very well-known reaction produces—due to the need of stoichiometric amounts—a significant amount of waste material at the end of the process. Also the chemical compounds which are used in such processes are not easy to handle.

Now the goal was to find an improved process for the ethynylation of the specific polyunsaturated aldehydes and ketones, which are represented by the following formula (I)

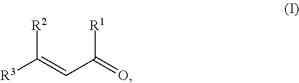

wherein

R¹ signifies —H, —CH₃ or —CH₂CH₃, and

R² signifies —H, —CH₃ or —CH₂CH₃, and

R³ signifies a moiety

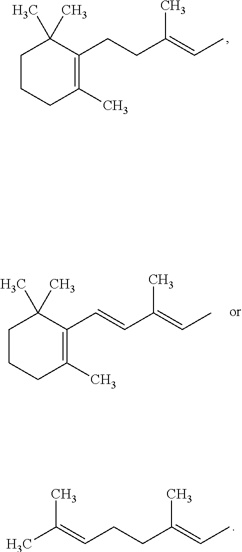

Surprisingly it was found that these specific (at least α,-β, unsaturated) polyunsaturated aldehydes and ketones can be ethynylated catalytically.

The ethynylation is carried out with ethyne (=acetylene; HCCH) under pressure by using at least one solvent and at least one catalyst.

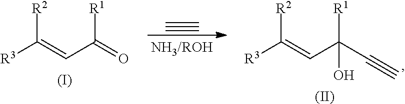

wherein

R¹, R², R³ are as defined above and

R is K or Cs, preferably K.

Therefore, the present invention relates to a catalytic ethynylation (CE) of compounds of formula (I)

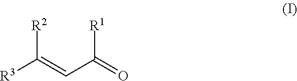

wherein

R¹ signifies —H, —CH₃ or —CH₂CH₃, and

R² signifies —H, —CH₃ or —CH₂CH₃, and

R³ signifies a moiety

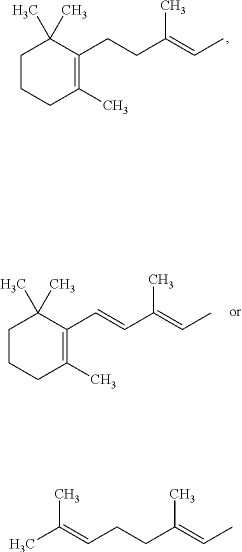

with ethyne

in the presence of a catalyst of formula ROH wherein

R signifies K or Cs, and wherein

NH₃ is used as solvent.

As stated above, the catalytic ethynylation of such important specific compounds of formula (I) are not known from the prior art.

This new method is an alternative way to for ethynylation, with significant advantages a catalytic process has (less starting material, less waste, safety of the process etc).

Preferred is a catalytic ethynylation as described above wherein formula (I)

R¹ signifies —H or —CH₃

R² signifies —H or —CH₃.

Therefore, the present invention relates to a catalytic ethynylation (CE1), which is ethynylation (CE), wherein formula (I)

R¹ signifies —H or —CH₃

R² signifies —H or —CH₃, preferably —H.

More preferred is the catalytic ethynylation of a compound of formula (Ia)

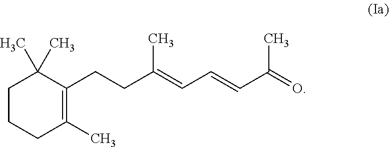 [SMILES: CC(=O)/C=C/C=C(\C)CCC1=C(C)CCCC1(C)C]

The corresponding ethynylated compound is the one of formula (IIa)

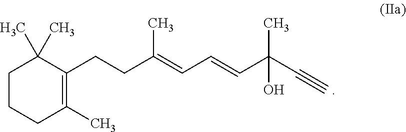 [SMILES: C#CC(C)(O)/C=C/C=C(\C)CCC1=C(C)CCCC1(C)C]

More preferred is the catalytic ethynylation of a compound of formula (Ib)

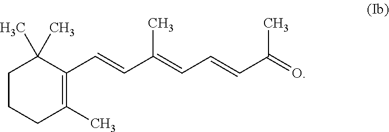 [SMILES: CC(=O)/C=C/C=C(C)/C=C/C1=C(C)CCCC1(C)C]

The corresponding ethynylated compound is the one of formula (IIb)

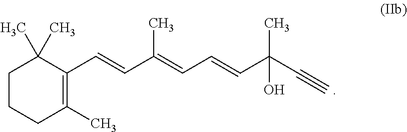 [SMILES: C#CC(C)(O)/C=C/C=C(C)/C=C/C1=C(C)CCCC1(C)C]

More preferred is the catalytic ethynylation of a compound of formula (Ic)

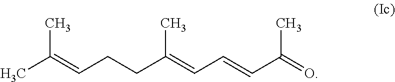

The corresponding ethynylated compound is the one of formula (IIc):

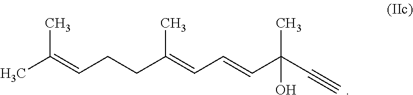 [SMILES: C#CC(C)(O)/C=C/C=C(\C)CCC=C(C)C]

Therefore, the present invention also relates to a catalytic ethynylation (CE2), which is ethynylation (CE), wherein the compound of formula (I) is the compound of formula (Ia)

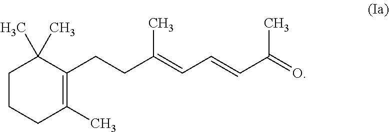 [SMILES: CC(=O)/C=C/C=C(\C)CCC1=C(C)CCCC1(C)C]

Therefore, the present invention also relates to a catalytic ethynylation (CE3), which is ethynylation (CE), wherein the compound of formula (I) is the compound of formula (Ib)

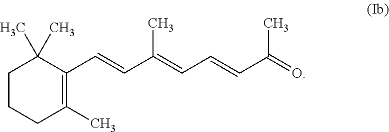 [SMILES: CC(=O)/C=C/C=C(C)/C=C/C1=C(C)CCCC1(C)C]

Therefore, the present invention also relates to a catalytic ethynylation (CE4), which is ethynylation (CE), wherein the compound of formula (I) is the compound of formula (Ic)

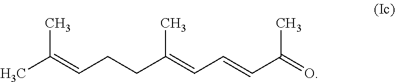

The catalyst (KOH or CsOH as well as mixture of these compounds) is usually added to the reaction mixture as an aqueous solution (30-60 wt-%, preferably 40-50 wt-%, based on the total weight of the aqueous catalyst solution).

It is preferred that the catalyst according to the present invention is KOH (R is K).

Therefore, the present invention also relates to a catalytic ethynylation (CE5), which is ethynylation (CE), (CE1), (CE2), (CE3) or (CE4), wherein R is K.

Therefore, the present invention also relates to a catalytic ethynylation (CE6), which is ethynylation (CE), (CE1), (CE2), (CE3), (CE4) or (CE5), wherein the catalyst is added to the reaction mixture as an aqueous solution (30-60 wt-%, of the catalyst, preferably 40-50 wt-%, based on the total weight of the aqueous catalyst solution).

The catalytic ethynylation according to the present invention is usually carried out under pressure. The pressure is in the range from 2 bar to 15 bar, preferably from 5 bar to 12 bar, more preferably from 6 bar to 10 bar.

Therefore, the present invention also relates to a catalytic ethynylation (CE7), which is ethynylation (CE), (CE1), (CE2), (CE3), (CE4), (CE5) or (CE6), wherein the pressure is in the range from 2 bar to 15 bar (preferably from 5 bar to 12 bar, more preferably from 6 bar to 10 bar).

The catalytic ethynylation according to the present invention is usually carried at a reaction temperature of between −40° C.-10° C., preferably −30°-5° C.

Therefore, the present invention also relates to a catalytic ethynylation (CE8), which is ethynylation (CE), (CE1), (CE2), (CE3), (CE4), (CE5), (CE6) or (CE7), wherein the process is carried at a reaction temperature of between −40° C.-10° C. (preferably −30°-5° C.).

The obtained reaction products are useful intermediates in organic synthesis, for example in the production of vitamin A (or vitamin A acetate) or carotenoids.

The following Examples illustrate the invention further without limiting it. All percentages and parts, which are given, are related to the weight and the temperatures are given in ° C., when not otherwise stated.

### EXAMPLES

### Example 1: Ethynylation of Pseudo-Ionone (Compound of Formula (Ic))

The reaction was carried out in a 1 L-autoclave, which was washed with N₂ before its use. Afterwards 150 g NH₃ was added into the autoclave and cooled to −20° C. At this temperature acetylene (ethyne) was added so that the pressure was 5.6 bara. Afterwards 1.68 g of KOH (as a 44 wt-% solution in H₂O) was added followed by 354.45 g of the compound of formula (Ic). After 4 h reaction time, the reaction mixture was diluted with 100 ml n-heptane.

The obtained product (compound of formula (IIc)

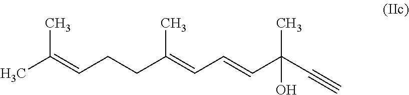 [SMILES: C#CC(C)(O)/C=C/C=C(\C)CCC=C(C)C]

was purified and it was obtained in a yield of 32%.

### Example 2: Ethynylation of Compound of Formula (Ia)

The reaction was carried out in a 1 L-autoclave, which was washed with N₂ before its use. Afterwards 150 ml of NH₃ was added and cooled to −20° C. At this temperature acetylene (ethyne) was added so that the pressure was 5.6 bara. 1.68 g of KOH (as a 44 wt-% solution in H₂O) was added.

Afterwards 5 g of the compound of formula (Ia), dissolved in 5 g toluene, was added. After 6 h reaction time, the reaction mixture was diluted with 100 ml toluene.

The obtained product (compound of formula (IIa)

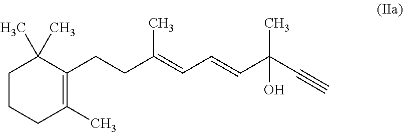 [SMILES: C#CC(C)(O)/C=C/C=C(\C)CCC1=C(C)CCCC1(C)C]

was purified and it was obtained in a yield of 57%.

1. A catalytic ethynylation of a compound of formula (I) 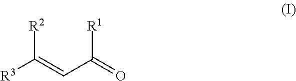
wherein
R¹ signifies —H, —CH₃ or —CH₂CH₃, and
R² signifies —H, —CH₃ or —CH₂CH₃, and
R³ signifies a moiety 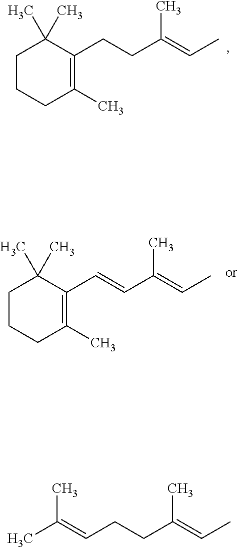
with ethyne
in the presence of a catalyst of formula ROH, wherein
R signifies K or Cs, and
wherein NH₃ is used as solvent.

2. Catalytic ethynylation according to claim 1, wherein
R¹ signifies —H or —CH₃, and
R² signifies —H or —CH₃, preferably —H.

3. Catalytic ethynylation according to claim 1, wherein the compound of formula (I) is the compound of formula (Ia)

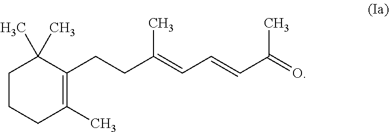 [SMILES: CC(=O)/C=C/C=C(\C)CCC1=C(C)CCCC1(C)C]

4. Catalytic ethynylation according to claim 1, wherein the compound of formula (I) compound of formula (Ib)

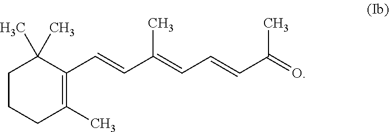 [SMILES: CC(=O)/C=C/C=C(C)/C=C/C1=C(C)CCCC1(C)C]

5. Catalytic ethynylation according to claim 1, wherein the compound of formula (I) compound of formula (Ic)

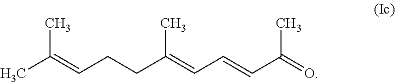

6. Catalytic ethynylation according to claim 1, wherein the catalyst is added to the reaction mixture as an aqueous solution.

7. Catalytic ethynylation according to claim 1, wherein R is K.

8. Catalytic ethynylation according to claim 1, wherein the catalytic ethynylation is carried out under pressure and wherein the pressure is in the range from 2 bar to 15 bar (preferably from 5 bar to 12 bar, more preferably from 6 bar to 10 bar).

9. Catalytic ethynylation according to claim 1, wherein the catalytic ethynylation is carried at a reaction temperature of between −40° C.-10° C. (preferably −30°-5° C.).
